(19) United States
(12) Patent Application Publication
(10) Pub. No.: US 2022/0277804 A1
(43) Pub. Date: Sep. 1, 2022

(54) SYSTEM AND METHODS FOR ELECTROSTATIC ANALYSIS WITH MACHINE LEARNING MODEL

(71) Applicant: BOARD OF TRUSTEES OF MICHIGAN STATE UNIVERSITY, East Lansing, MI (US)

(72) Inventors: Guowei WEI, Haslett, MI (US); Zixuan CANG, Irvine, CA (US); Jiahui CHEN, Dallas, TX (US); Weihua GENG, Dallas, TX (US)

(21) Appl. No.: 17/637,440

(22) PCT Filed: May 5, 2020
(86) PCT No.: PCT/US2020/031426

§ 371 Date: Feb. 22, 2022

Related U.S. Application Data
(60) Provisional application No. 62/890,976, filed on Aug. 23, 2019.

(51) Int. Cl. G16B 15/30; G16B 40/00; G16B 45/00; G06N 7/08; G06F 17/13

(52) U.S. Cl. CPC G16B 15/30; G16B 40/00; G16B 45/00; G06N 7/08; G06F 17/13

(57) ABSTRACT

Systems and methods are described relating to a Poisson Boltzmann machine learning model, which may be executed to predict electrostatic solvation free energy for molecular compounds, such as proteins. Feature data input to the Poisson Boltzmann machine learning model may include multi-weighted colored subgraph centralities, which may be calculated based on edge definitions of pairwise atomic interactions between atoms of a given protein using a generalized exponential function and/or a generalized Lorentz function, either or both of which may be weighted based on atomic rigidity or atomic charge. Predictions of electrostatic solvation free energy performed by the Poisson Boltzmann machine learning model may be used as a basis for ranking candidate compounds for a defined target clinical application.

### CROSS REFERENCE TO RELATED APPLICATIONS

This application claims priority to U.S. Provisional Application No. 62/890,976, filed Aug. 23, 2019, the content of which is incorporated herein by reference in its entirety.

### STATEMENT REGARDING FEDERALLY SPONSORED RESEARCH

This invention was made with government support under 1418957 and 1721024 awarded by the National Science Foundation, and under GM126189 awarded by the National Institutes of Health. The government has certain rights in the invention.

### BACKGROUND

Understanding the characteristics, activity, and structure-function relationships of biomolecules is an important consideration in modern analysis of biophysics and in the field of experimental biology. For example, these relationships are important to understanding how biomolecules react with one another (such as solvation free energies).

Electrostatics is ubiquitous in the molecular and biomolecular world. The analysis of molecular electrostatics is of crucial importance to the research community. There are two significant types of electrostatic analyses, namely, qualitative analysis for general electrostatic characteristics, such as visualization and electrostatic steering, and quantitative analysis for statistical, thermodynamic and/or kinetic observables, such as solvation free energy, solubility, and partition coefficient.

Molecular electrostatics can be analyzed by explicit or implicit models. Explicit solvent models, including molecular dynamics, resolve electrostatic effect in atomic detail and thus are more accurate but can be very expensive for large biomolecular systems. Implicit solvent models describe the solvent as a dielectric continuum, while the solute molecule is modeled with an atomistic description. A wide variety of two-scale implicit solvent models have been developed for electrostatic analysis, including generalized Born (GB), polarizable continuum, and Poisson-Boltzmann (PB) models. GB methods tend to be faster compared to PB methods but provide only heuristic estimates for electrostatic energies. PB methods, formally derived from the Maxwell theory, offer more accurate methods for electrostatic analysis, but tend to be slower than GB methods. It is desirable to develop accurate, efficient, reliable and robust PB solvers.

Mathematically, the PB equation is a nonlinear partial differential equation with point charges. Additionally, for biomolecules, the continuum-discrete interface is non-smooth, which gives rise to a nonlinear elliptic interface problem with discontinuous coefficients and singular source terms. Development of accurate, efficient and robust numerical solution for the PB equation in a biomolecular setting is notoriously challenging, Specifically, most conventional solution methods suffer from slow convergence due to the lack of the appropriate treatment of the highly complex biomolecular interface. In fact, the problem of the mathematical representation of the biomolecular interface is difficult to address with traditional methods.

The generation of highly accurate electrostatic potentials for large biomolecules can be extremely expensive with conventional methods. For example, it takes days to compute the electrostatic free energy of a protein with 50,000 atoms at the mesh of 0.2 Å on a single CPU using conventional PB solving methods. Additionally, the information generated for the electrostatic analysis of a given biomolecule is not translatable to other proteins. Therefore, with such conventional methods, one has to carry out the separated electrostatic analysis of different proteins or the same protein with different protonation states or conformations, which tends to result in a major consumption of community resources.

Additionally, a need exists for systems that can provide faster, less expensive, less invasive, more robust, and more humane tools for analyzing biologically-active compounds. For example, high throughput screening (HTS) for potential drug compounds is a multi-billion dollar global market, which is expanding greatly year over year due to a growing, and aging, population. HTS techniques are used for conducting various genetic, chemical, and pharmacological tests that aid the drug discovery process starting from drug design and initial compound assays, to supporting compound safety assessments leading to drug trials, and other necessary regulatory work concerning drug interactions. For compound screening, currently, one of the predominant techniques used is a 2-D cell-based screening technique that is slow, expensive, and can require detailed processes and redundancies to guard against false or tainted results. Automated approaches based on biomolecular models are limited in their use, due to the limitations of current techniques.

### BRIEF DESCRIPTION OF THE DRAWINGS

FIG. 1 illustrates an example of a solute disposed in a solvent, and boundaries and charges thereof, which may be represented via an implicit solvent model.

FIG. 2 shows an illustrative graph demonstrating differences in mean absolute percentage error (MAPE) across different grid sizes for two conventional Poisson Boltzmann solvers and a Poisson Boltzmann Machine Learning (PBML) model, in accordance with an embodiment.

FIG. 3A shows an illustrative graph demonstrating differences in mean CPU time per protein versus MAPEs for two conventional Poisson Boltzmann solvers and different instances of the PBML model having different mesh sizes, in accordance with an embodiment.

FIG. 3B shows a zoomed-in view of FIG. 3A over a smaller range of MAPE values.

FIG. 4 shows an illustrative process flow for a method by which electrostatic solvation free energy of a given protein may be predicted and stored, in accordance with an embodiment.

FIG. 5 shows an illustrative process flow for a method by which a multi-weighted colored subgraph (MWCS) centrality may be calculated for a given protein, in accordance with an embodiment.

FIG. 6 depicts an illustrative process flow for identifying a set of compounds based on a target clinical application, a set of desired characteristics, and a defined class of compounds, predicting characteristics of the set of compounds using trained machine learning models, ranking the set of compounds, identifying a subset of compounds based on the rankings, and synthesizing molecules of the subset of compounds, in accordance with example embodiments.

FIG. 7 is an illustrative block diagram of a computer system that may execute some or all of any of the methods of FIGS. 4, 5, and/or 6, in accordance with example embodiments.

FIG. 8 is an illustrative block diagram of a server cluster that may execute some or all of any of the methods of FIGS. 4, 5, and/or 6, in accordance with example embodiments.

### DETAILED DESCRIPTION

The present disclosure provides a machine learning based solution of the PB equation for the electrostatic analysis of molecules and biomolecules. A probability distribution of molecular and/or biomolecular electrostatics for a given compound (e.g., a given molecule within a given solvent) can be characterized by a mathematical representation of electrostatic potential. It is impractical, if not impossible, to perfectly represent the exact form of this probability distribution, even if the PB equation is solved for all possible molecular and/or biomolecular structures represented in the distribution. However, a PB solver may sample the probability distribution. One approach may be based on a representability hypothesis and a learning hypothesis. The representability hypothesis states that the electrostatic potential of a molecule or biomolecule can be described by a set of interactive charges and their geometric relations with a surrounding solvent. This hypothesis may be the basis for the construction of a feature vector for the characterization of the probability distribution of molecular and/or biomolecular electrostatics. The learning hypothesis essentially states that molecular and biomolecular electrostatics can be effectively represented by a feature vector as described by the representability hypothesis. When the probability distribution of molecular and/or biomolecular electrostatics of a compound is sufficiently sampled from a training set, a machine learning model can be established based on training labels and associated feature vectors as well as advanced machine learning algorithms to accurately predict the electrostatic potential of an unseen dataset which shares the same probability distribution with the training set.

However, the protocol described above with effective feature vectors and advanced machine learning algorithms does not guarantee the accuracy of the machine learning solution to the PB equation for molecular and biomolecular electrostatics. The caveat, more specifically, is the PB solver, whose accuracy determines the accuracy of the machine learning labels, and thus the estimated probability distribution of molecular and biomolecular electrostatics. There is no exact solution to the PB model for complex molecules and biomolecules. Nevertheless, the convergence of a given PB solver can be tested by a systematic mesh refinement. Essentially, the solution of an accurate PB solver does not change much during the mesh refinement, whereas that an inaccurate PB solver tends to change dramatically. Additionally, when the same set of PB parameters, (e.g., charge assignments, probe radii, and dielectric constants), are used, the solution of a less accurate PB solver would converge to that of a more accurate PB solver as the mesh is systematically refined. After the convergence test of various PB solvers, a machine learning model should be constructed with the solutions of the most accurate PB solver at a fine mesh. The accuracy of the machine learning model can access by a comparison of its predictions of a test set with the solutions of various PB solvers for the same test at different mesh sizes. In some embodiments, none of the molecules in the test set may be the same as anyone in the training set. An accurate machine learning model would show the convergence of the solutions of many PB solvers at various mesh sizes toward its predictions.

The representability hypothesis does not specify how to construct an accurate and efficient representation. An average biomolecule in the human body consists of about six thousands of atoms that lie in an 18,000-dimensional Euclidean space) ( [symbol] ^{18,000}). Such a high-dimensionality generally makes the first principle calculations intractable in practical large data predictions. Additionally, the direct use of three-dimensional (3D) macromolecular structures in deep convolutional neural networks (CNNs) tends to be extremely expensive and uncompetitive. For example, a brute force three-dimensional (3D) representation of biomolecules at a low resolution of 0.5 Å can give rise to a machine learning feature dimension of 100³n, where n is the number of element types. The variable sizes of biomolecules also tend to hinder the application of conventional machine learning algorithms. These challenges can be addressed by developing scalable and intrinsically low-dimensional representations of biomolecular structures. Intrinsic physics may lie in low-dimensional manifolds or spaces embedded in a high dimensional data space. Systems and methods described herein may utilize a graph theory representation of molecules due to its simplicity, in conjunction with results obtained from a GB model.

### Poisson-Boltzmann Model

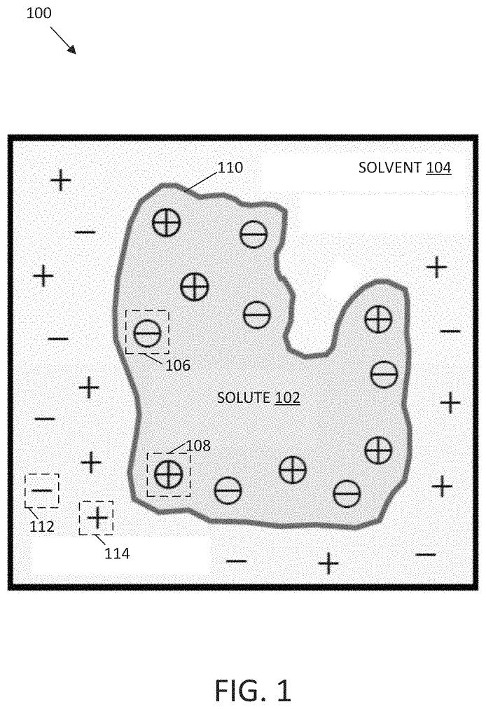

The PB model is a two-scale implicit solvent model that may be used for electrostatic analysis of molecular systems. FIG. 1 shows an illustrative compound 100 that may be described by a PB model. The compound 100 may be any molecular or biomolecular compound comprising a solvent and solute. The PB model describes the two-scale treatment of the electrostatics of the compound 100, with an interior domain Ω₁, corresponding to the region occupied by a solute biomolecule 102 having fixed positive charges 108 and negative charges 106. An exterior domain Ω₂, corresponding to the region occupied by a solvent 104 and positive ions 114 and negative ions 112 dissolved within the solvent, surrounds the solute biomolecule 102. An interface 110, which may be represented herein as Γ separates the biomolecular domain Ω₁ and solvent domain Ω₂. While a variety of surface models can be used, the most commonly used one is the solvent excluded surface or molecular surface. The biomolecule in domain Ω₁ consists of a set of atomic charges qₖ located at atomic centers rₖ for k=1, . . . , N_{c} with N_{c} as the total number of charges. In the solvent domain Ω₂, the charge source density of mobile ions 112 and 114 can be approximated by the Boltzmann distribution. For simplicity, a linearized PB model is considered in the present example according to EQ. 1:

−∇·ϵ(r)∇ϕ(r)+κ̅²(r)ϕ(r)=Σₖ₌₁^{N}^{_{c}}qₖδ(r−rₖ), EQ. 1

where ϕ(r) represents electrostatic potential of the compound 100, −∇ represents the divergence, and ϵ(r) represents the dielectric constant of the compound 100 given by EQ. 2:

$$ϵ ( r ) = \{ \begin{matrix}{ϵ}_{1} , & r ∈ {Ω}_{1} , \\ {ϵ}_{2} , & r ∈ {Ω}_{2} ,\end{matrix} \qquad EQ . 2$$

and κ̅ is the screening parameter with the relation κ̅²=ϵ₂κ² where κ is the inverse Debye length (e.g., the Debye length being a measure of a charge carrier's net electrostatic effect in a solution and how far its electrostatic effect persists) measuring the ionic effective length of the compound 100. Interface conditions on the molecular surface (e.g., at the interface 110) may be calculated according to EQ. 3:

$${ϕ}_{1} ( r ) = {ϕ}_{2} ( r ) , {ϵ}_{1} \frac{\partial {ϕ}_{1} ( r )}{\partial n} , {ϵ}_{2} \frac{\partial {ϕ}_{2} ( r )}{\partial n} , r ∈ Γ , \qquad EQ . 3$$

where ϕ₁ and ϕ₂ are the limit values when approaching the interface from the inside or outside the solute domain, and n is the outward unit normal vector on Γ. Inappropriate treatment of these interface conditions can be a source of error in conventional PB solvers. The far-field boundary condition for the PB model is provided in EQ. 4:

$${lim}_{\vert r \vert \to ∞} ϕ ( r ) = 0. \qquad EQ . 4$$

In practice, the far-field boundary condition may be implemented approximately. The electrostatic salvation free energy can be obtained from the PB model by EQ. 5:

$$Δ {G}_{elec}^{PB} = \frac{1}{2} {\sum }_{k = 1}^{{N}_{C}} {q}_{k} ( ϕ ( {r}_{k} ) - {ϕ}_{0} ( {r}_{k} ) ) , \qquad EQ . 5$$

where ϕ₀(rₖ) is the free space solution to the PB equation assuming no solvent-solute interface. To solve the PB equation, a second order PB solver based on a matched interface and boundary (MIB) method (sometimes referred to as a “MIBPB solver”) may be applied to interpolate ϕ(r) values from grid meshes to an atomic position whenever the corresponding atom is close to the interface Γ. ΔG_{elec}^{PB} results generated by the MIBPB solver for a set of macromolecules may be used as training labels when training the Poisson-Boltzmann Machine Learning (PBML) model described below.

### Generalized Born Model

The Generalized Born (GB) model is a fast approximation to the PB model. Compared to the PB model, the GB model offers a relatively simple and more computationally efficient approach to compute the long-range electrostatic interactions in biomolecules, which tends to be the bottleneck in classical all-atom simulations. With appropriate parameterization, a GB solver can be as accurate as a PB solver. The GB approximation of electrostatic solvation free energy can be expressed via EQ. 6:

$$Δ {G}_{elec}^{GB} \approx {\sum }_{i j} Δ {G}_{i j}^{G B} = - \frac{1}{2} ( \frac{1}{{ϵ}_{1}} - \frac{1}{{ϵ}_{2}} ) \frac{1}{1 + α β} {\sum }_{i j} q q ( \frac{1}{f ( {r}_{i j} , {R}_{i} , {R}_{j} )} + \frac{αβ}{A} ) , \qquad EQ . 6$$

where Rᵢ is the effective Born radius of a given atom i, rᵢⱼ is the distance between atoms i and j, β=ϵ₁/ϵ₂, α=0.57142, and A is the electrostatic size of the molecule. The function ƒᵢⱼ is represented in EQ. 7:

$${f}_{ij} = \sqrt{{r}_{i j}^{2} + {R}_{i} {R}_{j} exp ( - \frac{{r}_{i j}^{2}}{4 {R}_{i} {R}_{j}} )} . \qquad EQ . 7$$

The effective Born radii Rᵢ are calculated by the boundary integral defined in EQ. 8:

$${R}_{i}^{- 1} = {( \frac{1}{2} {∮}_{Γ} \frac{r - r}{{\vert r - r \vert}^{6}} \cdot dS )}^{1 / 3} . \qquad EQ . 8$$

To carry out the boundary integral, a triangulation discretization of the molecular surface may be generated and applied.

### Graph Theory Representation

Graph theory is a prime subject of discrete mathematics and concerns graphs as mathematical structures for modeling pairwise relations between vertices, nodes, or points. Such pairwise relations define graph edges. Algebraic graph theory, such as spectral graph theory, studies algebraic connectivity, characteristic polynomials, and eigenvalues and eigenvectors of matrices associated with a given graph, such as the adjacency matrix or Laplacian matrix. Graphs of this nature have applications in chemistry and biomolecular modeling. However, the diagonalization of the interaction Laplacian matrix has a high computational complexity per number of matrix elements. This time-consuming matrix diagonalization can be bypassed using geometric graph theory techniques.

In conjunction with machine learning algorithms and models, a multiscale weighted color subgraph (MWCS) technique may be used to represent complex biomolecular structures. In the present example, protein structures in particular will be considered. A weighted colored subgraph (WCS) may be generated to describe electrostatic interactions in a protein of N atoms. The WCS incorporates kernels to characterize pairwise distance-weighted atomic correlations. All interactions are classified according to element types, represented by colored subgraphs. To use a WCS to analyze protein electrostatic interactions, all atoms in the protein and their pairwise interactions may be formulated into a weighted graph G(V,E) with vertices V and edges E. As such, the ith atom is labeled by both its position rᵢ and its element type αᵢ. Vertices of the WCS can therefore be expressed according to EQ. 9:

V={(rᵢ, αᵢ)|rᵢ∈ [symbol] ³; αᵢ∈ [symbol] ; i=1, 2, . . . , N}, EQ. 9

where [symbol] ={C, N, O, S, H} contains all the commonly occurring element types in a protein. It should be noted that [symbol] may be modified for different (e.g., non-protein) biomolecular systems.

To describe pairwise interactions, a colored set [symbol] ={αβ} with α, β∈ [symbol] . With this setting, it can be verified that [symbol] , in the present example, has a total of 15 unordered element-wise partitions or subsets: {CC, CN, CO, CS, CH, NN, NO, NS, NH, OO, OS, OH, SS, SH, HH}. For each subset of element pairs [symbol] ₖ, k=1,2, . . . , 15, a set of vertices [symbol] is a subset of V containing all atoms that belong to the pair in [symbol] ₖ. For example, a partition [symbol] ₂={CN} contains all pairs of atoms in the protein with one atom being a carbon and another atom being a nitrogen. Based on this setting, all edges in such a WCS describing pairwise interactions may be defined by EQ. 10:

[symbol]

={Φ_{τ,ζ}^{σ(∥}rᵢ−rⱼ∥)|(αᵢβⱼ)∈ [symbol] ₖ; i=1, 2, . . . , N_{α}; j=1, 2, . . . , N_{β}}, EQ. 10

where ∥rᵢ−rⱼ∥ defines a Euclidean distance between the i^{th} and the j^{th} atoms, σ indicates the type of radial basic functions (e.g., L for Lorentz kernel, E for exponential kernel), τ is a scale distance factor between two atoms, and ζ is a parameter of power in the kernel (i.e., ζ=κ when σ=E, ζ=υ when σ=L). The kernel Φ_{τ,ζ}^{σ} characterizes a pairwise correlation satisfying the conditions of EQs. 11 and 12:

Φ_{τ,ζ}^{σ}(∥rᵢ−rⱼ∥)=1, as ∥rᵢ−rⱼ∥→0, EQ. 11

Φ_{τ,ζ}^{σ}(∥rᵢ−rⱼ∥)=0, as ∥rᵢ−rⱼ∥→∞, EQ. 12

Examples of radial basis functions include generalized exponential functions, defined in EQ. 13:

Φ_{τ,κ}^{E}(∥rᵢ−rⱼ∥)=e^{-(∥r}^{ᵢ}^{-r}^{ⱼ}^{∥/τ(r}^{ᵢ}^{+r}^{ⱼ}⁾⁾^{^{κ}}, κ>0, EQ. 13

and generalized Lorentz functions defined in EQ. 14:

$${Φ}_{τ , υ}^{L} ( ‖ {r}_{i} - {r}_{j} ‖ ) = \frac{1}{1 + {( ‖ {r}_{i} - {r}_{j} ‖ \text{/} τ ( {r}_{i} + {r}_{j} ) )}^{v}} , v > 0 , \qquad EQ . 14$$

where rᵢ and rⱼ are, respectively, the van der Waals radii of the i^{th} and the j^{th} atoms. Centrality may be used as a factor for representing node importance. The degree of centrality counts the number of edges of a node. For example, harmonic centrality can be regarded as an extension of the harmonic formulation per EQ. 15:

$${μ}_{i}^{k , σ , τ , v , w} = {\sum }_{j = 1}^{\vert {V}_{{𝒫}_{k}} \vert} {w}_{ij} {Φ}_{τ , v}^{σ} ( ‖ {r}_{i} - {r}_{j} ‖ ) , ( {α}_{i} {β}_{j} ) ∈ {𝒫}_{k} , ∀ i = 1 , 2 , … , \vert {V}_{{𝒫}_{k}} \vert , \qquad EQ . 15$$

where wᵢⱼ is a weight function assigned to each atomic pair. It can be chosen either as wᵢⱼ=1 for atomic rigidity or wᵢⱼ=qⱼ for atomic charge.

To describe a centrality for a whole MWCS, G( [symbol] , [symbol] ), the summation of the atomic centralities may be calculated according to EQ. 16:

$${μ}_{i}^{k , σ , τ , v , w} = {\sum }_{j = 1}^{\vert {V}_{{𝒫}_{k}} \vert} {μ}_{j}^{k , σ , τ , v , w} \qquad EQ . 16$$

It is this subgraph centrality that makes partition {CN} equivalent to partition {NC}. Since there are 15 choices from the colored subsets (i.e., subsets of weighted colored edges) [symbol] ₖ, we can obtain 15 sub-graph centralities for each μᵢ^{σ,τ,ν,ω}. By varying kernel parameters, multiscale centralities for MWCS can be achieved. For two-scale WCS (e.g., in two values [E, L] for σ and two values [1, q] for ω are represented across all 15 values of k), 60 descriptors can be obtained for a protein. The results of EQ. 16 can be used as features provided as inputs to a Poisson-Boltzmann Machine Learning (PBML) model.

The vertices V and the collection of all edges E defines a weighted graph G(V,E). However, G(V,E) alone may have limited descriptive power in machine learning prediction. Thus, MWCSs G( [symbol] , [symbol] ) and their centralities μ^{k,σ,τ,ν,ω} may be used as input features to a PBML to describe protein electrostatics.

### Poisson-Boltzmann Machine Learning (PBML) Model

The prediction of PB electrostatic solvation free energy may be formulated based on a supervised learning approach to create PBML model. A training data set may be expressed according to EQ. 17:

D={(x⁽ⁱ⁾,y⁽ⁱ⁾)|x∈ [symbol] ,y∈ [symbol] , i=1, . . . , M}. EQ. 17

where x⁽ⁱ⁾ is the feature vector for the ith sample in the training set y⁽ⁱ⁾=ΔG⁽ⁱ⁾ is the label (e.g., the electrostatic solvation free energy of the ith sample). Here, n is the length of the feature vector and M is the total number of samples in the training set. Since the electrostatic solvation free energy of a PB model cannot be obtained analytically, ΔG⁽ⁱ⁾ for the training set (e.g., the “true” electrostatic solvation free energy against which the predictions of the PBML model will be compared during training) may be calculated using an accurate PB solver, such as the MIBPB solver, which may be determined from a convergence analysis. The GB model and MWCS may be used to generate the feature vector for the training data set. A test data set may be similarly generated, and may be used to validate the PBML once it is trained. In the present example, a gradient boosting decision tree (GBDT) machine learning model may be used as the basis for the PBML model. While a GBDT model is described here, it should be understood that other machine learning algorithms, including linear regression, random forest, neural networks (e.g., deep neural networks) can be trained using the training data set D and then applied instead to predict electrostatic (e.g., solvation) free energy of the PB model.

As the GB model provides a good approximation to the PB model, ΔG_{elec}^{GB} can be incorporated into the machine learning algorithm utilized by the PBML model. A GBDT algorithm based on the GB model may be used to implement the PBML model, and will now be described.

To create the GBDT algorithm, a decision tree T₁ may be built to fit the training data set D, leading to predicted labels p₁ of EQ. 18:

{p₁(x⁽ⁱ⁾)}_{i<1}^{M}. EQ. 18

The errors (sometimes referred to as “residues”) r₂⁽ⁱ⁾ corresponding to the predictions p₁ are represented in EQ. 19:

r₂⁽ⁱ⁾=y⁽ⁱ⁾−p₁(x⁽ⁱ⁾). EQ. 19

If r₂⁽ⁱ⁾ does not equal 0 for some sample i, then another decision tree T₂ is built to fit the data set represented in EQ. 20:

{(x⁽ⁱ⁾, r₂⁽ⁱ⁾)}ᵢ₌₁^{M}, EQ. 20

leading to new predicted labels p₂ of EQ. 21

{p₂(x⁽ⁱ⁾)}ᵢ₌₁^{M}. EQ. 21

The errors corresponding to the predictions p₂ are represented in EQ. 22:

r₃⁽ⁱ⁾=r₂⁽ⁱ⁾−p₂(x⁽ⁱ⁾)=y⁽ⁱ⁾−p₁(x⁽ⁱ⁾)−p₂(x⁽ⁱ⁾), EQ. 22

and the predicted labels for the data set D are then represented in EQ. 23:

{p₁(x⁽ⁱ⁾)+p₂(x⁽ⁱ⁾)}ᵢ₌₁^{M}. EQ. 23

If r₃⁽ⁱ⁾ does not equal 0 for some sample i, then another decision tree T₃ is built to fit the data set represented in EQ. 24:

{(x⁽ⁱ⁾, r₃⁽ⁱ⁾)}ᵢ₌₁^{M}, EQ. 24

leading to new predicted labels p₃ of EQ. 25:

{p₃(x⁽ⁱ⁾)}ᵢ₌₁^{M}. EQ. 25

More generally, the predicted model for the training data set D based on K consecutive decision trees is represented in EQ. 26:

ŷ_{K}⁽ⁱ⁾=Σₖ₌₁^{K}pₖ(x⁽ⁱ⁾), i=1,2, . . . , M. EQ. 26

This is the so-called boosting tree procedure. A loss function may be defined according to EQ. 27:

Lₖ=Σᵢ₌₁^{M}lₖ(y⁽ⁱ⁾−ŷₖ⁽ⁱ⁾)=Σᵢ₌₁^{M}½(y⁽ⁱ⁾−ŷₖ⁽ⁱ⁾)², EQ. 27

which may minimize the loss via the gradient descent optimization of the decision trees. In order to minimize the loss, a gradient may first be calculated according to EQ. 28:

$${r}_{k}^{( i )} = - \frac{\partial {l}_{k - 1}}{\partial {p}_{k - 1} ( {x}^{( i )} )} \qquad EQ . 28$$

Decision trees Tₖ may then be constructed to fit the gradient, leading to predicted labels pₖ as the learner function of Tₖ. Finally, a learning rate αₖ may be chosen, as defined in EQ. 29:

αₖ:=argmin_{α}Σᵢ₌₁^{M}lₖ₋₁(y⁽ⁱ⁾−ŷₖ₋₁⁽ⁱ⁾)+αpₖ(x⁽ⁱ⁾), Eq. 29

which may then be used, in combination with a predefined shrinkage parameter to update the model according to EQ. 30:

ŷₖ⁽ⁱ⁾=ŷₖ₋₁⁽ⁱ⁾ηαpₖ(x⁽ⁱ⁾). EQ. 30

The features used as inputs to the PBML may include one or more representations of MWCS centrality, which may be calculated in accordance with some or all of EQs. 9-16. In some embodiments (e.g., in which the biomolecular compound being considered is a protein), the input features for a PBML may include MWCS centralities based on four different sets of kernel parameters: {μ^{k,E,τ,2,1}}ₖ₌₁¹⁵, {μ^{k,E,τ,2,q}}ₖ₌₁¹⁵, {μ^{k,L,τ,2,1}}ₖ₌₁¹⁵, and {μ^{k,L,τ,2,q}}ₖ₌₁¹⁵. In other words, the input features may include 60 different MWCS centralities with 15 being based on generalized exponential functions and an atomic rigidity weight function, 15 being based on generalized Lorentz functions and an atomic rigidity weight function, 15 being based on generalized exponential functions and an atomic charge weight function, and 15 being based on generalized Lorentz functions and an atomic charge weight function.

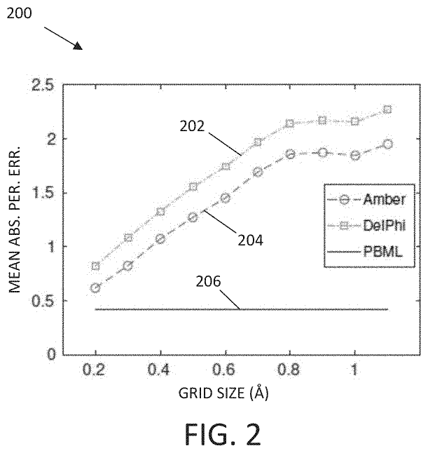

FIG. 2 shows a graph 200, which compares the mean absolute percentage errors (MAPEs) of predicted electrostatic solvation free energies of a test set of 195 proteins using three different methods: Amber (i.e., Amber PBSA), DelPhi, and the PBML method described above. Line 202 represents the MAPEs corresponding to the DelPhi method, which is a finite difference method (FDM) PB solver. Line 204 represents Amber method, which is another FDM PB solver. Line 206 represents the PBML method that corresponds to the present disclosure. The MAPEs are referenced against the results of another FDM PB solver, MIBPB, at a grid size of 0.2 Å. As shown, across mesh sizes ranging from 0.2 Å to 1 Å, the PBML method of predicting electrostatic solvation free energy consistently outperforms the Amber and DelPhi solvers.

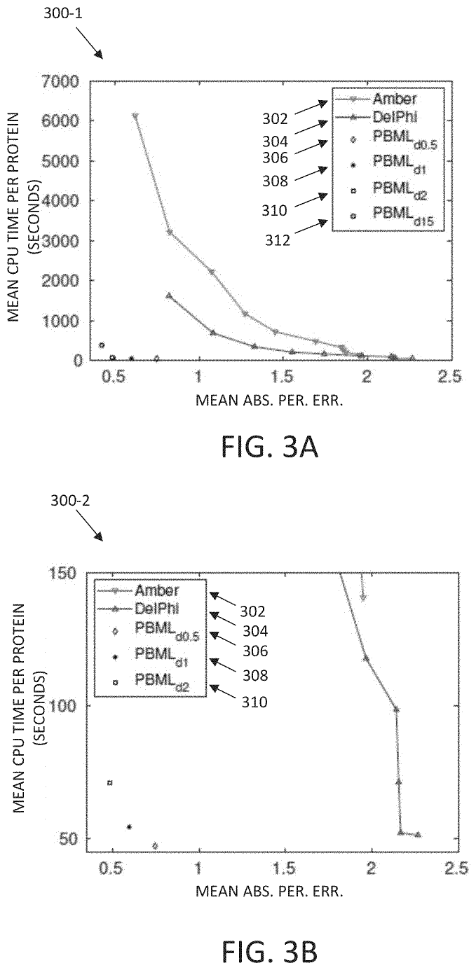

FIGS. 3A and 3B show graphs 3004 and 300-2 representing the MAPE vs. mean CPU time per protein in seconds for prediction of electrostatic solvation free energies using a test set of 195 proteins (e.g., the same test set of proteins used in FIG. 2) by the Amber method, DelPhi method, and four different instances of the PBML method corresponding to four different mesh densities (0.5, 1, 2, and 15) used to represent the biomolecular interface (e.g., the triangulation density of the MSMS surface). Line 302 represents the results of the Amber method, with each point on the line 302 representing a different mesh size from 0.2 Å to 1.1 Å. Line 304 represents the results of the DelPhi method with each point on the line 302 representing a different mesh size from 0.2 Å to 1.1 Å. Point 306 represents the results of the PBML method instance with mesh density 0.5. Point 308 represents the results of the PBML method instance with mesh density 1. Point 310 represents the results of the PBML method instance with mesh density 2. Point 312 represents the results of the PBML method instance with mesh density 15. It should be noted that graph 300-2 is a reduced-scale version of graph 300-1, showing the range of mean CPU time per protein from around 40 seconds to around 150 seconds.

As shown in graphs 300-1 and 300-2, each of the PBML method instances outperform the Amber and Delphi methods. Additionally, it should be noted that the MAPE of the PBML approach tends to decrease with decreased mesh density, whereas the mean CPU time per protein tends to increase with decreased mesh density.

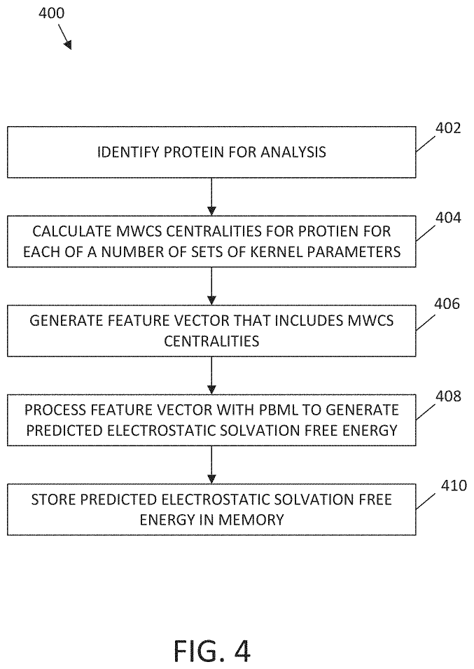

FIG. 4 shows an illustrative process flow for a method 400 by which electrostatic solvation free energy may be predicted using a PBML. The method 400 may be performed, for example, by executing computer readable instructions stored on a memory device with one or more computer processors (referred to collectively as “the processor” in the present example).

At step 402, the processor identifies a protein for analysis. For example, the processor may receive a request for prediction of electrostatic solvation free energy of a particular protein. The request may define the atoms of the protein and their relative atomic positions, in some embodiments. In other embodiments, the protein may be identified by name, and the processor may retrieve a listing of the atoms of the protein and their relative atomic positions from a corresponding data store.

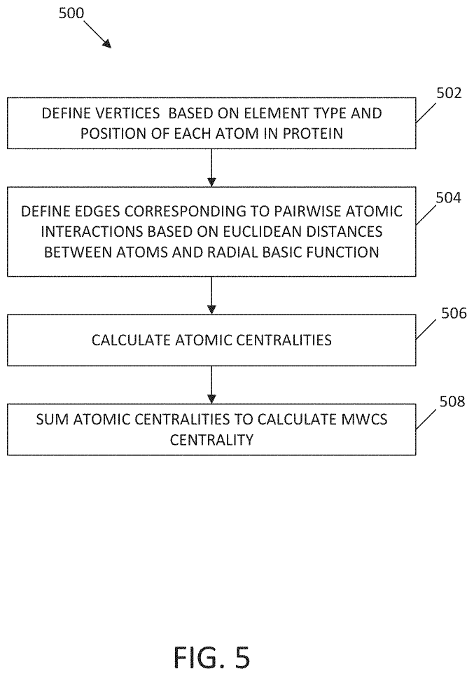

At step 404, the processor calculates MWCS centralities for each of a number of sets of kernel parameters. For example, these may include MWCS centralities based on four different sets of kernel parameters, each spanning 15 different atomic pairs: {μ^{k,E,τ,2,1}}ₖ₌₁¹⁵, {μ^{k,E,τ,2,q}}ₖ₌₁¹⁵, {μ^{k,L,τ,2,1}}ₖ₌₁¹⁵, and {^{k,L,τ,2,q}}ₖ₌₁¹⁵. An example of how a given MWCS centrality may be calculated is provided in FIG. 5.

FIG. 5 shows an illustrative process flow of a method 500 for calculating the MWCS centrality of a protein. While the present example focuses on MWCS centrality calculations for proteins, it should be understood that the method 500 may be applicable to other types of biomolecular compounds as well. The method 500 may be performed, for example, by executing computer readable instructions stored on a memory device with one or more computer processors (referred to collectively as “the processor” in the present example).

At step 502, the processor defines vertices for each of the atoms in the protein based on the element type and position of each atom. For example, the vertices may be defined according to EQ. 9.

At step 504, the processor defines edges corresponding to pairwise atomic interactions between atoms of the protein using a radial basic function based on the Euclidean distances between pairs of atoms of the protein. For example, the edges may be defined according to EQ. 10, and the radial basic function may be defined according to EQ. 13 when a generalized exponential function is used, and may be defined according to EQ. 14 when a generalized Lorentz function is used.

At step 506, the processor calculates atomic centralities for each atom in the protein. For example, atomic centralities may be calculated according to EQ. 15.

At step 508, the processor calculates the MWCS centrality for the MWCS graph G(E,V) representation of the protein. For example, the MWCS centrality may be calculated according to EQ. 16.

Returning to FIG. 4, at step 406, the processor generates a feature vector that includes the MWCS centralities.

At step 408, the processor executes a trained PBML (e.g., which may be a GBDT-based PBML as described above) to process the feature vector to generate a predicted electrostatic solvation free energy of the protein (e.g., when in a solvent).

At step 410, the processor stores the predicted electrostatic solvation free energy in a memory device that is electrically (or otherwise communicatively) coupled to the processor.

### EXAMPLE—MONTE CARLO ANALYSIS

A major difficulty in molecular dynamics is the long timescales associated with real molecular processes taking place in nature. Ignoring the requirement of having time-resolved trajectories of molecular processes would remove such difficulty. It is generally sufficient to rely on a predicted representative ensemble of structures for a given process, which may be generated via Monte Carlo sampling.

The Monte Carlo methods are stochastic techniques that use random numbers to sample conformation space by iteratively generating random conformations (sometimes referred to herein as “configurations”) and either accepting or rejecting or accepting such conformations to gradually approach an expected result. The Monte Carlo methods may be applied via a Monte Carlo simulation (e.g., Metropolis's Monte Carlo simulation), by which predicted molecular states of a given molecule (e.g., biomolecule) may be generated according to Boltzmann probabilities (i.e., the Boltzmann distribution). Under a physiological condition, biomolecules are immersed and interact with surrounding solvent (e.g., water) molecules and interact with surrounding solvent molecules and other co-factors. The Monte Carlo simulation of such a biomolecule must therefore deal with a large number of solvent molecules, which can make such simulation very expensive, resource intensive, and, sometimes, intractable. Additionally, in Monte Carlo simulations, the biomolecular conformation may be subject to random perturbations, which generally result in overlaps between the biomolecule and explicit solvent molecules, which may lead to an unfavorable and non-representative structure. Implicit solvent models, such as the PB and GB methods, may overcome this challenge by taking a mean field approximation of water molecules and producing a dielectric continuum. As described above, a Poisson-Boltzmann machine learning (PBML) model can be applied to compute the solvation free energy of macromolecules within a solvent. The PBML model can, for example, be applied to predict the electrostatic solvation free energy portion of molecular solvation free energies in implicit-solvent Monte Carlo simulations with improved accuracy and speed compared to conventional Monte Carlo algorithms. It should be understood that molecular solvation free energy is the sum of electrostatic salvation free energy (i.e., ΔG_{elec}^{GB}) and nonpolar energy that represents the energy in the reversible work needed to insert a fixed configuration molecule into the solvent with all solute charges set to zero.

### Molecular Force Fields

Molecular force fields (e.g., a collection of equations and associated constraints designed to reproduce molecular geometry and selected properties of tested structures) offer a physical representation of molecular interactions and energy distributions. The quality of a molecular simulation is generally dependent upon the accuracy of the molecular force fields on which the simulation is based. Molecular force fields typically describe molecular interactions in terms of classical molecular mechanics of atoms. The potential energies of atomic interactions are approximated by a set of mathematical functions, modeling the bonded and non-bonded components. These functions consist of a set of free coefficients, which are obtained by approximating either the results of elaborate quantum mechanical calculations, or experimental data. One of the advantages of the biomolecular force field approach is its computational efficiency. The potential energy of a biomolecule can be efficiently computed at the molecular level using this approach. Additionally, the forces in molecular dynamics can be evaluated analytically from molecular force fields.

Methods described herein are generally considered in the context of the Amber force filed (e.g., the Amber ff99SB force field), defined according to EQ. 31:

$$E = {\sum }_{bonds} {{k}_{b} ( r - {r}_{0} )}^{2} + {\sum }_{angles} {{k}_{θ} ( θ - {θ}_{0} )}^{2} + … \\ {\sum }_{dihedrals} {V}_{n} [ 1 + cos ( n ϕ - γ ) ] + {\sum }_{i = 1}^{N - 1} {\sum }_{j = i + 1}^{N} [ \frac{{A}_{ij}}{{R}_{ij}^{12}} - \frac{{B}_{ij}}{{R}_{ij}^{6}} + \frac{{q}_{i} {q}_{j}}{{ϵ}_{1} {R}_{ij}} ] \qquad EQ . 31$$

where k_{b}, k_{θ}, and Vₙ are force constants. Here, r, θ, and ϕ are bond length, angle, and dihedral angle, respectively, while r₀, θ₀, γ represent optimal bond length, optimal angle, and proper dihedral angle, respectively. The first three terms in the energy expression describe the bonded energy of the molecular system, while the last term represents the Lennard-Jones interactions and electrostatic interactions, where N is the number of atoms in the molecular system, Rᵢⱼ is the distance between the ith and jth atoms, Aᵢⱼ and Bᵢⱼ are Lennard-Jones parameters, qᵢ is the atom charge and ϵ₁ is the dielectric constant.

### Monte Carlo Methods

Moving on to the computation of actual physical properties of a solute-solvent system using molecular dynamics, the classical expression of the partition function Q of a solute-solvent system is represented in EQ. 32:

$$Q = c \int drdp exp [ \frac{- ℋ ( r , p )}{{k}_{B} T} ] \qquad EQ . 32$$

where r={X,Y} stands for the atomic coordinates of a solute X and a solvent Y, p stands for the corresponding momenta, c is a physical constant, k_{B} is the Boltzmann constant, T is the temperature of the system, and [symbol] (r,p) is the Hamiltonian of the system (e.g., which describes the total energy of an individual system as a summation of the kinetic energy and the potential energy of that system). It can be assumed that all of the other physical observables A of interest depend only on the positions (i.e., A=A(r)). Therefore, the integration over the momenta, c, can be carried out analytically in a classical mechanical treatment. As a result, the expected value of a physical observable of interest (e.g., solvation free energy) can be given by EQ. 33:

$$〈 A 〉 = \frac{\int drA ( r ) exp [ - β E ( r ) ]}{\int dr exp [ - β E ( r ) ]} \qquad EQ . 33$$

where β=1/k_{B}T. To evaluate (A) conventionally requires numerical techniques such as Simpson's rule or the Trapezoidal rule for the integral. Since each particle moves in 3D space, the total number of degrees of freedom is 3N for a system of N atoms. If each dimension is integrated with a mesh size of in points, the total number of points for the integration is m^{3N}, which is computationally prohibitive.

The complexity in evaluating EQ. 33 can be significantly reduced using Monte Carlo sampling. The probability density function P(r) for finding a microstate in the canonical ensemble in a configuration r may be denoted by EQ. 34:

$$P ( r ) = \frac{exp [ - β E ( r ) ]}{\int dr exp [ - β E ( r ) ]} . \qquad EQ . 34$$

According to the probability function of EQ. 34, randomly selected points in the configuration can be perturbed. Hence, the number of points nᵢ generated per unit volume in the area around r is equal to N_{mc}P(r) for the average of A(r), which can be expressed by EQ. 35:

$$〈 A 〉 = \frac{1}{{N}_{mc}} {\sum }_{i = 1}^{{N}_{mc}} {n}_{i} A ( {r}_{i} ) . \qquad EQ . 35$$

EQ. 35 shows that all states of the ensemble contribute to the average equally. Therefore, the Metropolis Monte Carlo method starts at a given configuration initially the “current configuration”) r₀={X₀, Y₀} and next perturbs the configuration by a defined transformation with a new configuration r₁={X₁, Y₁}. The probability to accept the new configuration, p_{acc}, is defined in EQ. 36:

p_{acc}=min(1, exp(−β(E(r₀)−E(r₁))))). EQ. 36

If the new configuration is accepted, then the new configuration becomes the current configuration. If the new configuration is rejected, the previous configuration is retained (i.e., continues to be the “current configuration”). The process iterates until the iteration number equals a fixed number (e.g., which may be tracked by a counter stored in a memory device of the system that is incremented each time an iteration is performed, where the counter is compared to a predetermined value, and if the counter matches the predetermined value, the process ends). The structure will approach the Boltzmann distribution if the perturbations satisfy the condition defined in EQ. 37:

π(rᵢ)pᵢⱼ=π(rⱼ)pⱼᵢ, EQ. 37

where π(rᵢ) is the probability of the system in configuration rᵢ, where π(rⱼ) is the probability of the system in configuration rⱼ, where rᵢⱼ is the probability to perturb the configuration from state rᵢ to state rⱼ, and where pⱼᵢ is the probability to perturb the configuration from state rⱼ to state rᵢ.

The PBML model defined via EQs. 1-16 may be applied to predict the ΔG_{elec} (or, more specifically ΔG_{elec}^{GB}) component of molecular solvation free energy when applying a Monte Carlo method to reconstruct a molecule in a solvent, and may significantly reduce computation time needed to apply the Monte Carlo method compared to conventional techniques. Specifically, using results of an existing PB solver (e.g., finite difference method based solvers such as Amber PBSA, Delphi, APBS, MIBPB, or CHARMM PBEQ) as labels, and GB and MWCS results as features, a gradient boosting decision tree (GBDT) based PBML model can be trained and then utilized to predict solvation free energy prediction. The trained PBML model may then be applied during Monte Carlo analysis of a molecule in a solvent. For example, the trained PBML model may be used to predict the solvation free energy that is added to the total energy E in EQ. 36. It should be noted that the Monte Carlo steps may depend mainly on the number of atoms of the molecule(s) being simulated and the magnitudes of the perturbations of the configuration. For example, when simulating a large number of atoms, more time steps may be required than when simulating a molecule having a smaller number of atoms. For example, the amount by which the configuration is perturbed may at least partially determine the number of steps required for the Monte Carlo simulation, In some embodiments, the Monte Carlo simulation may be automatically stopped in response to a determination by the system that the error has fallen below a predetermined threshold or within a predetermined range.

### EXAMPLE—DRUG DISCOVERY

In some embodiments, the machine learning algorithms and associated methods of biomolecular analysis described above may have particular applications for the discovery of new drugs for clinical applications.

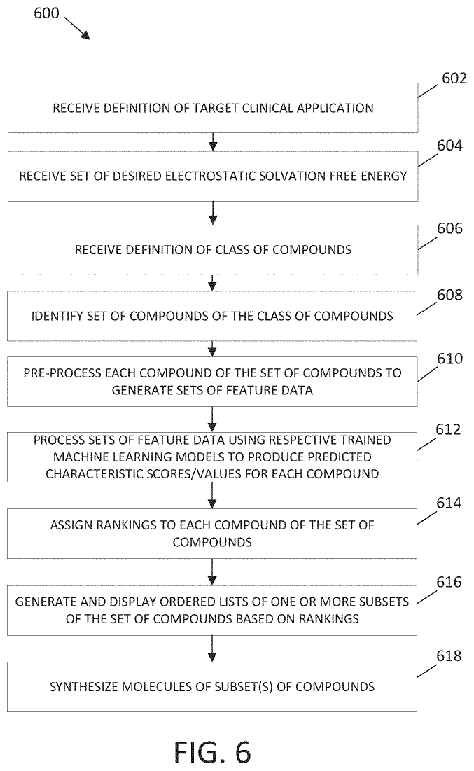

An illustrative example is provided in FIG. 6, which shows a flow chart for a method 600 by which one or more biomolecular models (e.g., complexes and/or dynamical systems, which may be represented by one or more datasets) representing biomolecular compounds (e.g., which may be limited to a particular class of compounds, in some embodiments) may be processed using one or more machine learning, implicit solvent, and/or graph theory algorithms or models to predict characteristics (e.g., solvation free energy) of each of the biomolecular compounds. The predicted characteristics may be compared between compounds and/or to predetermined thresholds, such that a compound or group of compounds that are predicted to most closely match a set of desired characteristics may be identified using the method 600. For example, the method 600 may be performed, at least in part, by executing computer-readable instructions stored on a non-transitory computer-readable storage device using one or more computer processors of a computer system or group of computer systems.

At step 602, a target clinical application is defined (e.g., via user input to the computer system(s)) and received by the computer system(s). For example, the target clinical application may correspond to a lead drug candidate to be discovered from among a class of candidate compounds and tested, Or, the target clinical application may simply involve performing predictions of certain characteristics of a specific small molecule or a complex macromolecule, for example. Thus, in a sense, the target clinical application could be user requesting a certain molecular analysis be conducted (a prediction of electrostatic solvation free energy).

At step 604, an electrostatic solvation free energy parameter may be defined (e.g., via user input) and received by the computer system(s). The electrostatic solvation free energy parameter may be a particular value or range of values of electrostatic solvation free energy that would be exhibited by a drug candidate that would be applicable for the target clinical application. In other words, the electrostatic solvation free energy parameter may correspond to a value or range of values of electrostatic solvation free energy that are desirable for the defined target clinical application. Thus, the electrostatic solvation free energy parameter may be referred to herein as the “desired” electrostatic solvation free energy. In the instance where the target clinical application is a request for a certain molecular analysis (e.g., rather than identification of candidate compounds for drug discovery), this step may be optional.

At step 606, a general class of compounds (e.g., biomolecules) is defined that are expected to exhibit the desired electrostatic solvation free energy. In some embodiments, the defined class of compounds may be defined manually via user input to the computer system. In other embodiments, the defined class of compounds may be determined automatically based on the defined target clinical application and the desired electrostatic solvation free energy. Alternatively, a specific compound may be identified, rather than a class of compounds, so that the specific compound can be the subject of the specified molecular analysis.

At step 608, a set of compounds (e.g., mathematical models/datasets of compounds) of the defined class of compounds is identified. In some embodiments, the set of compounds may be identified automatically based on the defined class of compounds, the electrostatic solvation free energy, and the target application. In other embodiments, the set of compounds may be input manually. For embodiments in which the set of compounds are input manually, steps 602, 604, and/or 606 may be optional.

At step 610, the set of compounds (or the specifically-identified individual compound) may be pre-processed to generate sets of feature data. For example, the pre-processing of the set of compounds may include performing graph theory (e.g., MWCS) calculations, determining MWCS centralities, and/or calculating/identifying auxiliary features for each compound. The sets of feature data may be generated for each compound using feature vectors calculated based on the MWCS centralities, and/or the auxiliary features for that compound, for example.

At step 612, the sets of feature data may be provided as inputs to and processed by a set of trained machine learning algorithms/models. For example, the set of trained machine learning algorithms/models may include the GBDT model described above, although other machine learning techniques could be used. For example, other applicable trained machine learning algorithm/models that may be included in the set of trained machine learning algorithms/models may include GBRT algorithms, naïve Bayes classification algorithms, ordinary least squares regression algorithms, logistic regression algorithms, support vector machine algorithms, other ensemble method algorithms, clustering algorithms (e.g., including neural networks), principal component analysis algorithms, singular value decomposition algorithms, autoencoder, generative adversarial network, recurrent neural network, short-long term memory, reinforcement learning, and independent component analysis algorithms. The trained machine learning algorithms/models may be trained to predict (e.g., quantitatively) properties of the input compounds with respect to the desired electrostatic solvation free energy. Moreover, the particular machine learning algorithm may be trained using a supervised training wherein feature data of only a particular class of molecules (e.g., only small molecules, or only proteins) are used, or multiple classes of molecules may be selected, so that the algorithm may have better predictive power for a given class of molecules. E.g., a machine learning algorithm may be selected that has been trained to be useful for proteins (e.g., trained using a training data set corresponding primarily or entirely to protein molecules), if the target class of compounds are proteins.

For each compound of the set of compounds, a value or score may be output by the trained machine learning model as a result of processing, the value or score corresponding to a predicted electrostatic solvation free energy value or a score representing how close (e.g., in value) a predicted electrostatic solvation free energy of the compound is to the desired electrostatic solvation free energy.

At step 614, the compounds of the set of compounds may then be assigned a ranking according to predicted score/value output for each compound, Each compound may receive a rankings for electrostatic solvation free energy, with compounds that have a predicted electrostatic solvation free energy that is closest to the desired electrostatic solvation free energy being listed first, and compounds that have a predicted electrostatic solvation free energy that is furthest from the desired electrostatic solvation free energy being listed last.

At step 616, the ordered list of compounds may be displayed (e.g., via an electronic display of the system), according to their assigned rankings. In some embodiments, only a subset of the ordered list may be displayed. As a non-limiting example, the subset of compounds may include only a predetermined number (e.g., 5) of the compounds having the highest rankings (e.g., the closest-to-desired predicted electrostatic solvation free energies), and may omit the remainder of the compounds.

At step 618, one or more molecules of the compounds having high or the highest rankings (e.g., those molecules included in the displayed subset of compounds) may be synthesized in order to begin applying these compounds in various trials. As an example, when initiating trials for a given compound of the subset of compounds, the given compound may be applied first in one or more in vitro tests (e.g., testing in biological matter for activity). Next, the given compound may be applied in one or more in vivo tests (e.g., testing in animals for toxicity, plasma binding affinity, pharmacokinetics, pharmacodynamics, etc.) if the outcomes of the in vitro tests were sufficiently positive. Finally, the given compound may be applied in a clinical trial in humans if the outcomes of the in vitro tests were sufficiently positive (e.g., showed sufficient efficacy, safety, and/or tolerability).

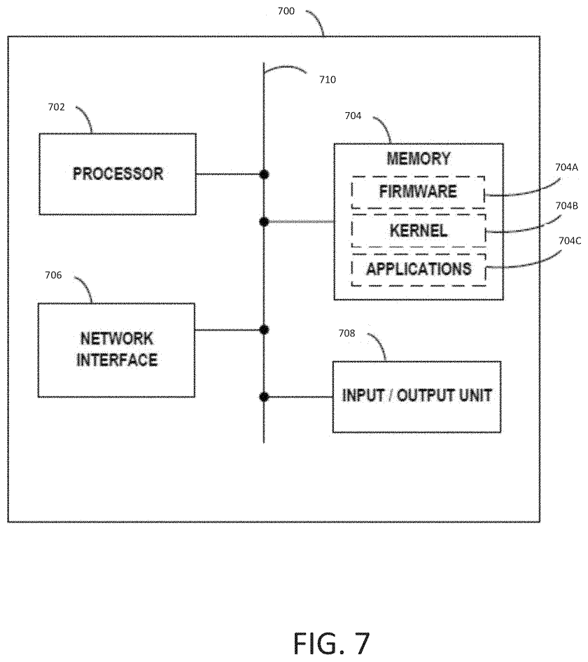

FIG. 7 is a simplified block diagram exemplifying a computing device 700, illustrating some of the components that could be included in a computing device arranged to operate in accordance with the embodiments herein. Computing device 700 could be a client device (e.g., a device actively operated by a user), a system or server device (e.g., a device that provides computational services to client devices), or some other type of computational platform. Some server devices may operate as client devices from time to time in order to perform particular operations, and some client devices may incorporate server features. The computing device 700 may, for example, be used to execute (e.g., via the processor 702 thereof) may be configured to execute, in whole or in part, any of the methods 400, 500, and 600 of FIGS. 4, 5, and 6.

In this example, computing device 700 includes processor 702., memory 704, network interface 706, and an input/output unit 708, all of which may be coupled by a system bus 710 or a similar mechanism. In some embodiments, computing device 700 may include other components and/or peripheral devices (e.g., detachable storage, printers, and so on).

Processor 702 may be one or more of any type of computer processing element, such as a central processing unit (CPU), a co-processor (e.g., a mathematics, graphics, or encryption co-processor), a digital signal processor (DSP), a network processor, and/or a form of integrated circuit or controller that performs processor operations. In some cases, processor 702 may be one or more single-core processors. In other cases, processor 702 may be one or more multi-core processors with multiple independent processing units. Processor 702 may also include register memory for temporarily storing instructions being executed and related data, as well as cache memory for temporarily storing recently-used instructions and data.

Memory 704 may be any form of computer-usable memory, including but not limited to random access memory (RAM), read-only memory (ROM), and non-volatile memory. This may include flash memory, hard disk drives, solid state drives, re-writable compact discs (CDs), re-writable digital video discs (DVDs), and/or tape storage, as just a few examples. Computing device 700 may include fixed memory as well as one or more removable memory units, the latter including but not limited to various types of secure digital (SD) cards. Thus, memory 704 represents both main memory units, as well as long-term storage, Other types of memory may include biological memory.

Memory 704 may store program instructions and/or data on which program instructions may operate. By way of example, memory 704 may store these program instructions on a non-transitory, computer-readable medium, such that the instructions are executable by processor 702 to carry out any of the methods, processes, or operations disclosed in this specification or the accompanying drawings.

As shown in FIG. 18, memory 704 may include firmware 704A, kernel 704B, and/or applications 704C. Firmware 704A may be program code used to boot or otherwise initiate some or all of computing device 700. Kernel 704B may be an operating system, including modules for memory management, scheduling and management of processes, input/output, and communication. Kernel 704B may also include device drivers that allow the operating system to communicate with the hardware modules e.g., memory units, networking interfaces, ports, and busses), of computing device 700. Applications 704C may be one or more user-space software programs, such as web browsers or email clients, as well as any software libraries used by these programs. Memory 704 may also store data used by these and other programs and applications.

Network interface 706 may take the form of one or more wireline interfaces, such as Ethernet (e.g. Fast Ethernet, Gigabit Ethernet, and so on). Network interface 706 may also support communication over one or more non-Ethernet media, such as coaxial cables or power lines, or over wide-area media, such as Synchronous Optical Networking (SONET) or digital subscriber line (DSL) technologies. Network interface 706 may additionally take the form of one or more wireless interfaces, such as IEEE 802.11 (Wifi), BLUETOOTH®, global positioning system (GPS), or a wide-area wireless interface. However, other forms of physical layer interfaces and other types of standard or proprietary communication protocols may be used over network interface 706. Furthermore, network interface 706 may comprise multiple physical interfaces. For instance, some embodiments of computing device 700 may include Ethernet, BLUETOOTH®, and Wifi interfaces.

Input/output unit 708 may facilitate user and peripheral device interaction with example computing device 700. Input/output unit 708 may include one or more types of input devices, such as a keyboard, a mouse, a touch screen, and so on. Similarly, input/output unit 708 may include one or more types of output devices, such as a screen, monitor, printer, and/or one or more light emitting diodes (LEDs). Additionally or alternatively, computing device 700 may communicate with other devices using a universal serial bus (USB) or high-definition multimedia interface (HDMI) port interface, for example.

In some embodiments, one or more instances of computing device 700 may be deployed to support a clustered architecture The exact physical location, connectivity, and configuration of these computing devices may be unknown and/or unimportant to client devices. Accordingly, the computing devices may be referred to as “cloud-based” devices that may be housed at various remote data center locations.

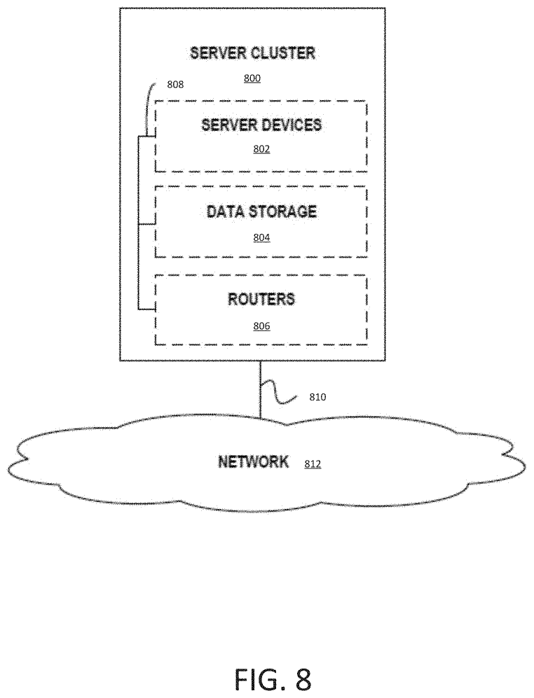

FIG. 8 depicts a cloud-based server cluster 800 in accordance with example embodiments. In FIG. 8, operations of a computing device (e.g., computing device 700) may be distributed between server devices 802, data storage 804, and routers 806, all of which may be connected by local cluster network 808. The number of server devices 802, data storages 804, and routers 806 in server cluster 800 may depend on the computing task(s) and/or applications assigned to server cluster 800 (e.g. the execution and/or training of machine learning models and/or algorithms, the calculation of feature data such as MWCSs, and other applicable computing tasks/applications). The server cluster 800 may, for example, be configured to execute (e.g., via computer processors of the server devices 802 thereof), in whole or in part, any of the methods 400, 500, and 600 of FIGS. 4, 5, and 6.

For example, server devices 802 can be configured to perform various computing tasks of computing device 700. Thus, computing tasks can be distributed among one or more of server devices 802. To the extent that these computing tasks can be performed in parallel, such a distribution of tasks may reduce the total time to complete these tasks and return a result. For purpose of simplicity, both server cluster 800 and individual server devices 802 may be referred to as a “server device.” This nomenclature should be understood to imply that one or more distinct server devices, data storage devices, and cluster routers may be involved in server device operations.

Data storage 804 may be data storage arrays that include drive array controllers configured to manage read and write access to groups of hard disk drives and/or solid state drives, The drive array controllers, alone or in conjunction with server devices 802, may also be configured to manage backup or redundant copies of the data stored in data storage 804 to protect against drive failures or other types of failures that prevent one or more of server devices 802 from accessing units of cluster data storage 804. Other types of memory aside from drives may be used.

Routers 806 may include networking equipment configured to provide internal and external communications for server cluster 800. For example, routers 806 may include one or more packet-switching and/or routing devices (including switches and/or gateways) configured to provide (i) network communications between server devices 802 and data storage 804 via cluster network 808, and/or (ii) network communications between the server cluster 800 and other devices via communication link 810 to network 812.

Additionally, the configuration of cluster routers 806 can be based at least in part on the data communication requirements of server devices 802 and data storage 804, the latency and throughput of the local cluster network 808, the latency, throughput, and cost of communication link 810, and/or other factors that may contribute to the cost, speed, fault-tolerance, resiliency, efficiency and/or other design goals of the system architecture.

As a possible example, data storage 804 may include any form of database, such as a structured query language (SQL) database. Various types of data structures may store the information in such a database, including but not limited to tables, arrays, lists, trees, and tuples. Furthermore, any databases in data storage 804 may be monolithic or distributed across multiple physical devices.

Server devices 802 may be configured to transmit data to and receive data from cluster data storage 804. This transmission and retrieval may take the form of SQL queries or other types of database queries, and the output of such queries, respectively. Additional text, images, video, and/or audio may be included as well. Furthermore, server devices 802 may organize the received data into web page representations. Such a representation may take the form of a markup language, such as the hypertext markup language (HTML), the extensible markup language (XML), or some other standardized or proprietary format, Moreover, server devices 802 may have the capability of executing various types of computerized scripting languages, such as but not limited to Python, PHP Hypertext Preprocessor (PHP), Active Server Pages (ASP), javaScript, and/or other languages such as C++, C#, or Java. Computer program code written in these languages may facilitate the providing of web pages to client devices, as well as client device interaction with the web pages.

The present invention has been described in terms of one or more preferred embodiments, and it should be appreciated that many equivalents, alternatives, variations, and modifications, aside from those expressly stated, are possible and within the scope of the invention.

1. A system comprising:
a non-transitory computer-readable memory; and
a processor configured to execute instructions stored on the non-transitory computer-readable memory which, when executed, cause the processor to:
identify a set of compounds based on one or more of a defined target clinical application, a set of desired characteristics, and a defined class of compounds;
pre-process each compound of the set of compounds to generate respective sets of feature data;
process the sets of feature data with a trained Poisson-Boltzmann machine learning model to produce a plurality of predicted electrostatic solvation free energies for each compound of the set of compounds, wherein the sets of feature data include multi-weighted colored subgraph centralities;
identify a subset of the set of compounds based on the plurality of predicted electrostatic solvation free energies; and
display an ordered list of the subset of the set of compounds via an electronic display.

2. The system of claim 1, wherein the instructions, when executed, further cause the processor to:
assign rankings to each compound of the set of compounds, wherein assigning a ranking to a given compound of the set of compounds for a given characteristic of the set of desired characteristics comprises:
comparing a first predicted electrostatic solvation free energy, corresponding to the given compound to other predicted electrostatic solvation free energies of other compounds of the set of compounds, wherein the ordered list is ordered according to the assigned rankings.

3. The system of claim 1, wherein the set of compounds includes proteins, and wherein the instructions, when executed, further cause the processor to, for a first protein of the proteins:
calculate a plurality of multi-weighted colored subgraph centralities for the first protein;
generate a feature vector that includes the multi-weighted colored subgraph centralities, wherein one of the sets of feature data includes the feature vector; and
process the feature vector with the Poisson-Boltzmann machine learning model to generate a predicted electrostatic solvation free energy of the first protein.

4. The system of claim 3, wherein, to calculate a first multi-weighted colored subgraph centrality of the plurality of multi-weighted colored subgraph centralities for the first protein, the instructions, when executed, cause the processor to:
define vertices for atoms of the first protein;
define first edges corresponding to pairwise atomic interactions between the atoms of the first protein using a generalized Lorentz function;
calculate first atomic centralities for each of the atoms of the first protein; and
sum the first atomic centralities to generate the first multi-weighted colored subgraph centrality.

5. The system of claim 4, wherein, to calculate a second multi-weighted colored subgraph centrality of the plurality of multi-weighted colored subgraph centralities for the first protein, the instructions, when executed, cause the processor to:
define second edges corresponding to pairwise atomic interactions between the atoms of the first protein using a generalized exponential function;
calculate second atomic centralities for each of the atoms of the first protein; and
sum the second atomic centralities to generate the second multi-weighted colored subgraph centrality.

6. The system of claim 5, wherein the generalized exponential function and the generalized Lorentz function are weighted based on atomic rigidity.

7. The system of claim 5, wherein the generalized exponential function and the generalized Lorentz function are weighted based on atomic charge.

8. A method comprising:
calculating, by a processor, a plurality of multi-weighted colored subgraph centralities for a protein;
generating, by the processor, a feature vector that includes the multi-weighted colored subgraph centralities; and
executing, by the processor, a Poisson-Boltzmann machine learning model to process the feature vector to generate a predicted electrostatic solvation free energy of the protein,

9. The method of claim 8, further comprising:
calculating, by the processor, a second plurality of multi-weighted colored subgraph centralities for a second protein;
generating, by the processor, a second feature vector that includes the second multi-weighted colored subgraph centralities; and
executing, by the processor, the Poisson-Boltzmann machine learning model to process the second feature vector to generate a second predicted electrostatic solvation free energy of the second protein;

10. The method of claim 9, further comprising:
assigning, by the processor, rankings to the protein and the second protein based on the first predicted electrostatic solvation free energy and the second predicted electrostatic solvation free energy; and
generating, by the processor, an ordered list that includes the protein and the second protein based on the rankings; and
causing, by the processor, the ordered list to be displayed at a user device.

11. The method of claim 10, further comprising:
calculating, by the processor, a first multi-weighted colored subgraph centrality of the plurality of multi-weighted colored subgraph centralities for the protein by:
defining, by the processor, vertices for atoms of the protein;
defining, by the processor, first edges corresponding to pairwise atomic interactions between the atoms of the protein using a generalized Lorentz function;
defining, by the processor, first atomic centralities for each of the atoms of the protein; and
summing, by the processor, the first atomic centralities to generate the first multi-weighted colored subgraph centrality.

12. The method of claim 11, further comprising:
calculating, by the processor, a second multi-weighted colored subgraph centrality for the protein by:
defining, by the processor, second edges corresponding to pairwise atomic interactions between the atoms of the protein using a generalized exponential function;
calculating, by the processor, second atomic centralities for each of the atoms of the protein; and
summing by the processor, the second atomic centralities to generate the second multi-weighted colored subgraph centrality.

13. The method of claim 12, wherein the generalized exponential function and the generalized Lorentz function are weighted based on atomic rigidity.

14. The method of claim 12, wherein the generalized exponential function and the generalized Lorentz function are weighted based on atomic charge.

15. A system comprising:
a non-transitory computer-readable memory; and
a processor configured to execute instructions stored on the non-transitory computer-readable memory which, when executed, cause the processor to:
receive an identifier corresponding to a protein;
generate feature data corresponding to the protein; and
process the feature data with a trained Poisson-Boltzmann machine learning model to produce a predicted electrostatic solvation free energy of the protein.

16. The system of claim 15, wherein the instructions, when executed, further cause the processor to:
calculate multi-weighted colored subgraph centralities for the protein;
generate a feature vector that includes the multi-weighted colored subgraph centralities, wherein the feature data includes the feature vector; and
process the feature vector with the Poisson-Boltzmann machine learning model to generate the predicted electrostatic solvation free energy of the protein.

17. The system of claim 16, wherein, to calculate a first multi-weighted colored subgraph centrality of the multi-weighted colored subgraph centralities, the instructions, when executed, cause the processor to:
define vertices for atoms of the protein;
define first edges corresponding to pairwise atomic interactions between the atoms of the protein using a generalized Lorentz function;
calculate first atomic centralities for each of the atoms of the protein; and
sum the first atomic centralities to generate the first multi-weighted colored subgraph centrality.

18. The system of claim 17, wherein, to calculate a second multi-weighted colored subgraph centrality of the multi-weighted colored subgraph centralities, the instructions, when executed, cause the processor to:
define second edges corresponding to pairwise atomic interactions between the atoms of the protein using a generalized exponential function;
calculate second atomic centralities for each of the atoms of the protein; and
sum the second atomic centralities to generate the second multi-weighted colored subgraph centrality.

19. The system of claim 18, wherein the generalized exponential function and the generalized Lorentz function are weighted based on atomic rigidity.

20. The system of claim 18, wherein the generalized exponential function and the generalized Lorentz function are weighted based on atomic charge.
